(19) United States
(12) Patent Application Publication
(10) Pub. No.: US 2025/0001181 A1
(43) Pub. Date: Jan. 2, 2025

(54) CLOSED-LOOP NEURAL INTERFACE FOR PAIN CONTROL

(71) Applicant: New York University, NEW YORK, NY (US)

(72) Inventors: Jing WANG, New York, NY (US); Zhe Sage CHEN, New York, NY (US)

(21) Appl. No.: 18/705,788

(22) PCT Filed: Nov. 2, 2022
(86) PCT No.: PCT/US2022/048714

§ 371 Date: Apr. 29, 2024

Related U.S. Application Data
(60) Provisional application No. 63/263,738, filed on Nov. 8, 2021.

(51) Int. Cl. A61N 1/36; A61N 1/02; A61N 1/05; A61N 1/375; A61N 5/06

(52) U.S. Cl. CPC A61N 1/36139; A61N 1/025; A61N 1/0534; A61N 1/36071; A61N 1/37514; A61N 5/0622; A61N 2005/0612; A61N 2005/0626

(57) ABSTRACT

A system and a method are for treating pain. The system includes a plurality probes implantable in multiple brain regions of a patient to detect neural signals including local field potentials of the multiple brain regions; a processing device receiving the neural signals from the multiple brain regions of a patient brain to process the neural signals and input the processed neural signals to a machine learning pain decoder model that is configured to indicate pain; and a stimulation device implantable in a target region of the patient brain to provide stimulation of the target region based upon an indication of pain.

### PRIORITY CLAIM

The present application is a National Phase Application of PCT Patent Application Serial No. PCT/US2022/048714 filed on Nov. 2, 2022; which claims priority to U.S. Provisional Application Ser. No. 63/263,738 filed on Nov. 8, 2021, and entitled, “Closed-Loop Neural Interface for Pain Control,” the disclosure of which is incorporated herewith by reference.

### GOVERNMENT FUNDING

This invention was made with government support under Grant No. NIH-R01GM115384 awarded by The National Institutes of Health. The government has certain rights in the invention.

### BACKGROUND

Chronic pain is one of the most common sensory disorders, and non-opioid and non-addictive analgesics are critically needed. Chronic pain is defined by discrete episodes of pain that are either evoked by noxious stimuli or occur spontaneously. Current treatment options for chronic pain are limited to either scheduled pharmacological interventions or continuous spinal or peripheral nerve neuromodulation. These therapeutic options do not take into consideration the precise timing of individual pain episodes and result in frequent delays in treatment as well as under or over treatment. Conceptually, a closed-loop neuromodulation approach that links timely pain detection with treatment is ideally suited to pain management by selectively targeting discrete nociceptive episodes. However, decoding pain signals remains an extremely challenging task. As pain has both sensory and affective dimensions, decoding pain should ideally combine both sensory and affective signals in the brain. Yet pain signals are encoded in multiple brain circuits. A second challenge is that most neuroimaging techniques are impractical for closed-loop use due to the large size and expense (e.g., MRI). Whereas invasive neural recording has the capacity for mobile and closed-loop applications, it typically does not yield consistent spike signals that facilitate sensory decoding. Solving these challenges are critical to the success if accurate pain decoding.

Meanwhile, in terms of treatment, deep brain stimulation (DBS) has been used to treat a number of neuropsychiatric conditions. However, constant stimulation has multiple drawbacks including desensitization leading to weaning of efficacy over time, battery consumption, and side effects. In addition, a closed-loop DBS system for treating pain has yet to be achieved.

### SUMMARY

The present disclosure relates to a computer-implemented method for detecting and treating chronic pain. The method includes receiving neural signals from multiple brain regions of a patient brain via probes implanted in the multiple brain regions, the neural signals including local field potentials (LFP) of the multiple brain regions; processing the neural signals and inputting the processed neural signals to a machine learning pain decoder model; determining, based on the processed neural signals, whether pain is indicated; and triggering, when pain is indicated via the pain decoder model, a stimulation of a target region of the patient brain based on an indication of the pain.

In an embodiment, processing the neural signals includes computing frequency dependent power features of the local field potentials of the multiple brain regions.

In an embodiment, determining whether pain is indicated includes identifying relative changes in neural activity in the multiple brain regions.

In an embodiment, the multiple brain regions include an anterior cingulate cortex and a primary somatosensory cortex.

In an embodiment, the stimulation of the target region of the patient brain includes an optical stimulation and an electrical stimulation.

In an embodiment, the target region of the patient brain includes a prefrontal cortex.

In an embodiment, the target region of the patient brain includes one of a primary motor cortex, an anterior cingulate cortex, and or periaqueductal gray and thalamus.

In an embodiment, the method further includes training the pain decoder model using a state space model based on spectral features from low gamma (30-50 Hz), high gamma (50-100 Hz), and ultra-high frequency (300-500 Hz) bands.

In addition, the present disclosure relates to a system for treating pain. The system includes a plurality of probes implantable in multiple brain regions of a patient to detect neural signals including local field potentials of the multiple brain regions; a processing device receiving the neural signals from the multiple brain regions of a patient brain to process the neural signals and input the processed neural signals to a machine learning pain decoder model that is configured to indicate pain; and a stimulation device implantable in a target region of the patient brain to provide stimulation of the target region based upon an indication of pain.

In an embodiment, the processing device is configured to process the neural signals by computing frequency dependent power features of the local field potentials of the multiple brain regions.

In an embodiment, the pain decoder model is trained to identify relative changes in neural activity in the multiple brain regions.

In an embodiment, the pain decoder model is trained using a state space model based on spectral features from low gamma (30-50 Hz), high gamma (50-100 Hz), and ultra-high frequency (300-500 Hz) bands.

In an embodiment, the processing device is configured to trigger activation of the stimulation device upon an indication of pain.

In an embodiment, the stimulation device is configured to provide one of optical and electrical stimulation of the target region.

In an embodiment, the stimulation device is configured to be implanted in one of a prefrontal cortex, a primary motor cortex, an anterior cingulate cortex, a periaqueductal gray, and thalamus.

In an embodiment, the plurality of probes is configured to be implanted in the multiple brain regions include an anterior cingulate cortex and a primary somatosensory cortex.

In an embodiment, each of the plurality of probes include a silicon probe array.

In an embodiment, the system further includes a graphical user interface displaying LFP signals in real-time and providing options to change threshold criterion.

Furthermore, the present disclosure relates to anon-transitory computer-readable storage medium including a set of instructions executable by a processor, the set of instructions, when executed by the processor causing the processor to perform operations, comprising: receiving neural signals from multiple brain regions of a patient, the neural signals including local field potentials (LFP) of the multiple brain regions; computing frequency dependent power features of the local field potentials of the multiple brain regions; inputting the power features to a machine learning pain decoder model to identify relative changes in neural activity in the multiple brain regions to indicate pain; and triggering stimulation of a target region of a brain based on an indication of pain.

### BRIEF DESCRIPTION

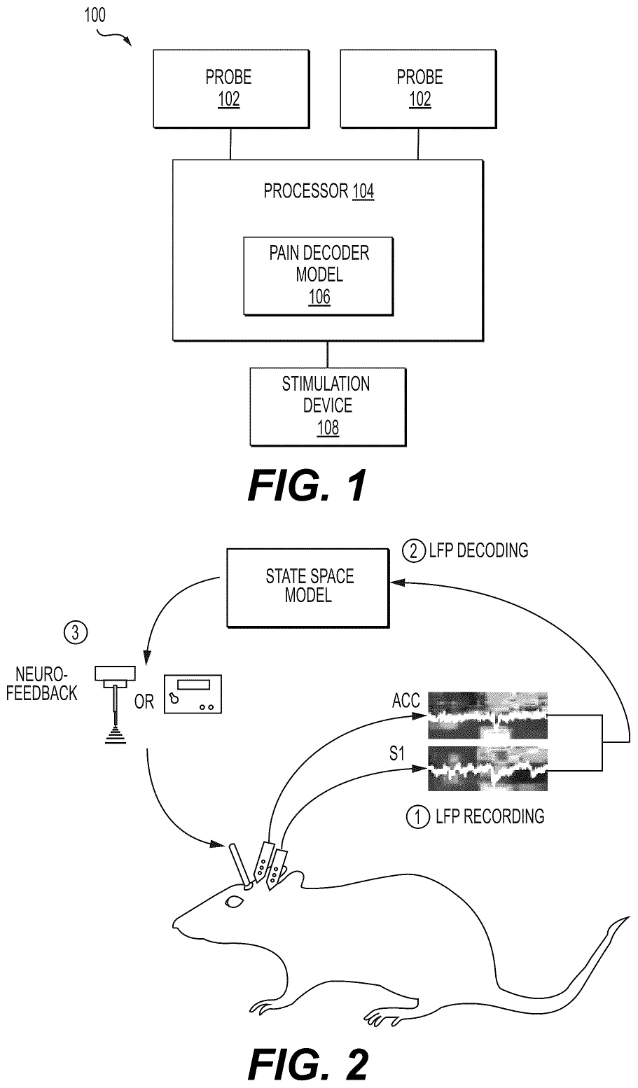

FIG. 1 shows a schematic diagram of a multi-region LFP-based Brain Machine Interface (BMI) system according to an exemplary embodiment of the present disclosure.

FIG. 2 shows a schematic diagram of an experiment based on the closed-loop BMI system according to the system of FIG. 1.

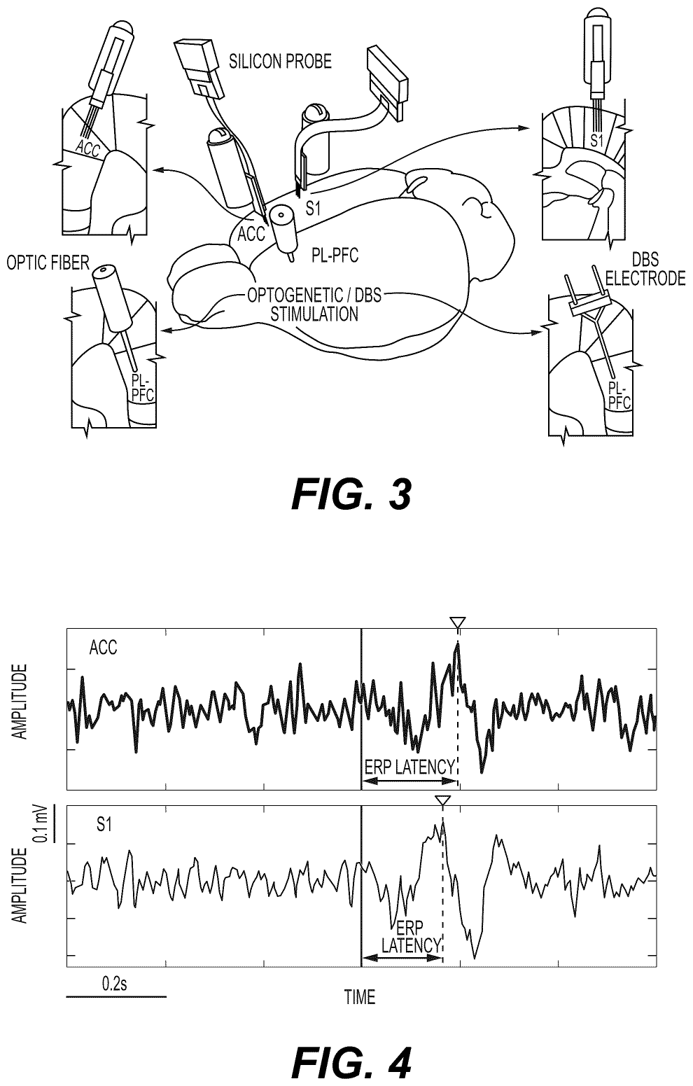

FIG. 3 shows a schematic diagram of a placement of the optic fiber or stimulating electrode in the PL-PFC and recording electrodes in the ACC and S1 according to the system of FIG. 1.

FIG. 4 shows an illustration of concurrent LFP signals in the ACC and S1, wherein pain evokes event-related potentials (ERPs) are marked by black triangles.

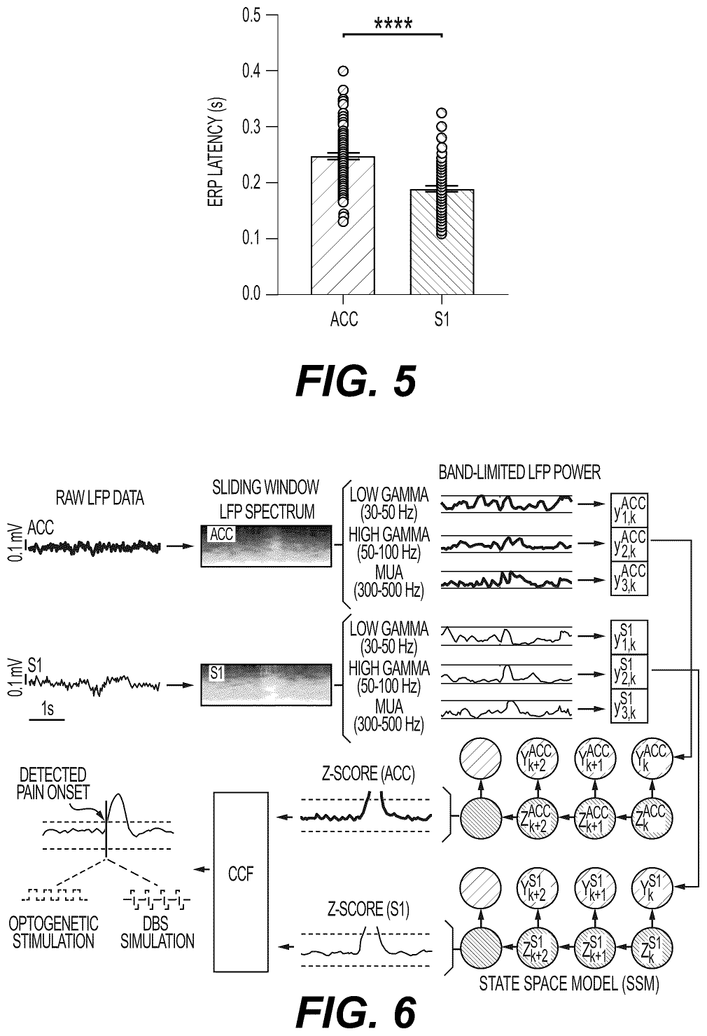

FIG. 5 shows a graph comparing ERP latency between the ACC and S1.

FIG. 6 shows a schematic diagram of a model-based unsupervised learning approach designed to decode pain from multi-region LFP signals according to the exemplary system of FIG. 1.

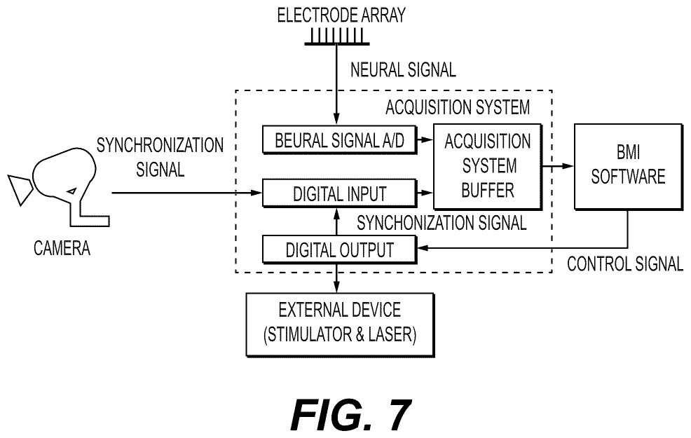

FIG. 7 shows a schematic diagram of an exemplary embodiment for a closed-loop multi-region LFP-based BMI.

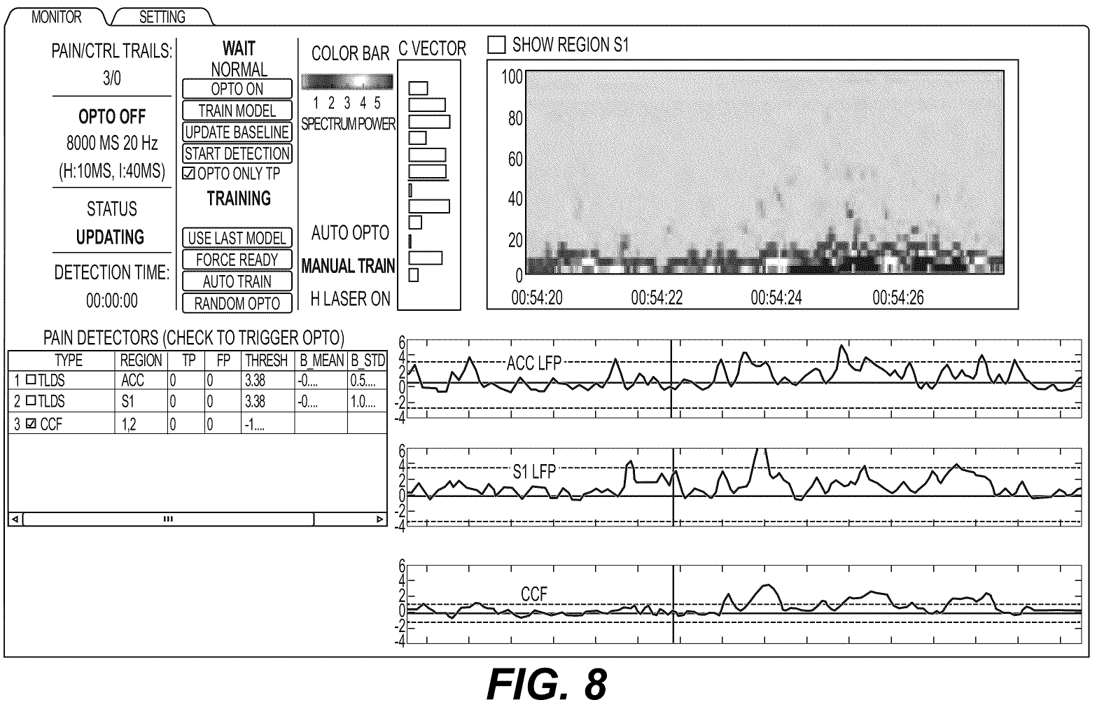

FIG. 8 shows an exemplary embodiment of a graphical user interface (GUI) of an exemplary BMI.

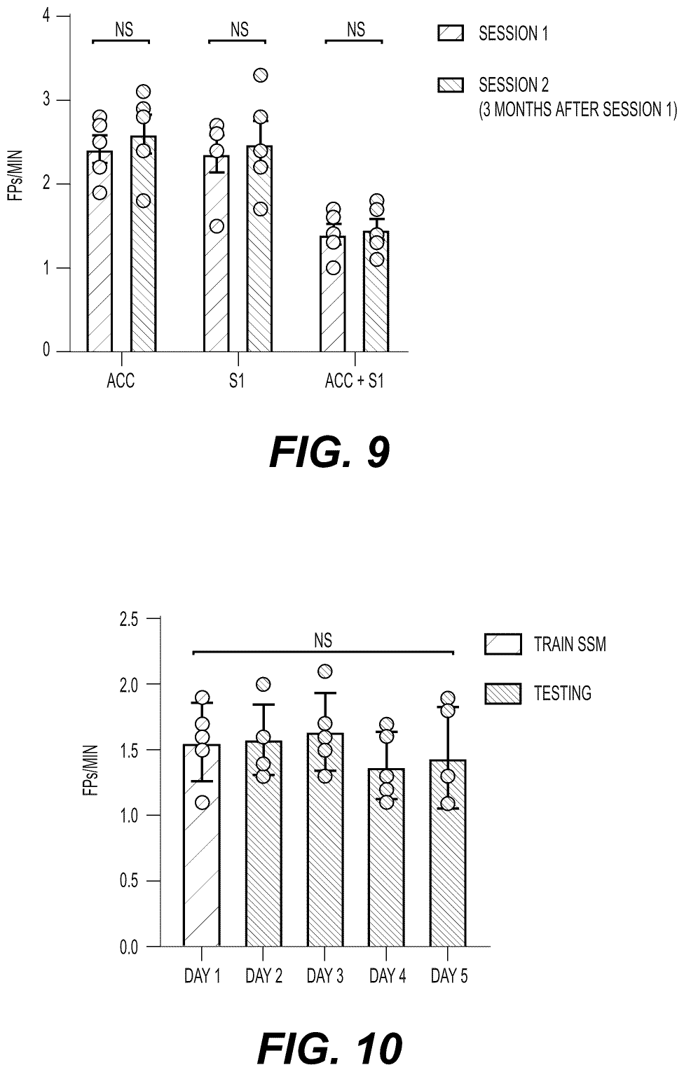

FIG. 9 shows a graph illustrating a comparison of false detection rates based on LFP decoding strategies.

FIG. 10 shows a graph illustrating a comparison of false detection rates based on model parameters set five days apart.

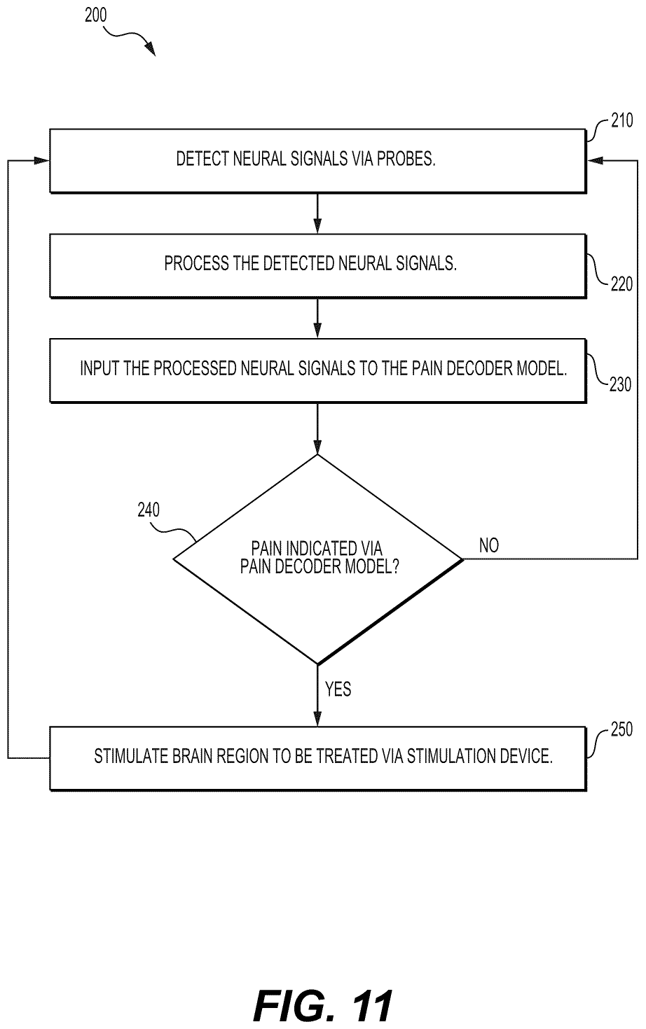

FIG. 11 shows a flow chart of a method for detecting and treating pain using a closed-loop multi-region LFP-based BMI system according to an exemplary embodiment of the present disclosure.

### DETAILED DESCRIPTION

The present disclosure may be further understood with reference to the following description and the appended drawings. Exemplary embodiments describe multi-region, closed loop neural interface for nociceptive control using a pain decoder based on concurrent neural signals from at least two critical regions for pain processing to trigger therapeutic stimulation of, for example, the prefrontal cortex. In an embodiment, the two brain regions include the anterior cingulate cortex (ACC) and primary somatosensory cortex (S1). The ACC is known to provide affective signal for pain, whereas the S1 contains the sensory pain signal. It has been shown that PFC stimulation can produce pain relief by activating multiple downstream pain-regulatory circuits. To decode pain onset, exemplary embodiments describe recording local field potentials (LFP), which represent subthreshold local neural activity and are stable in chronic electrophysiological recordings, which facilitates clinical application. A model-based, unsupervised machine learning pain decoder is trained to decode pain from multi-region LFP signal.

In an exemplary embodiment, the pain decoder focuses specifically on spectral features from low gamma (30-50 Hz), high gamma (50-100 Hz), and ultra-high frequency (300-500 Hz) bands. Frequency-dependent LFP power features are computed and inputted to a real-time neural pain decoder based on, for example, a state space model (SSM). In the presence of a noxious stimulus, the SSM identifies a relative change in observed neural activity (Z-scored) in the ACC or S1 from the baseline, and uses this change in neural activity as a proxy for the acute pain signal. In order to optimize the specificity of pain detection, in one embodiment, a cross-correlation function (CCF) may temporally track coherent changes of pain-encoded LFP features in the S1 and ACC, as the user of concurrent signals from the cortical regions capture both sensory and aversive components of pain to enhance pan decoding accuracy. This CCF combines the two SSM-inferred Z-scores derived separately from the SCC and S1 LFP features, and optimizes the detection performance by adjusting the relative weights of each region's contributions. For online closed-loop application, the CCF-based decoder may automatically detect the onset of nociceptive signal to trigger electrical stimulation of the PFC to treat pain.

The exemplary embodiments describe a closed-loop neural interface for pain control which automatically detects and treats pain with negligible delay. No human intervention is necessary. Pain detection is driven by simultaneous and/or concurrent recordings from multiple brain regions to enhance sensitivity and specificity. LFPs are used to decode pain, which provide stable signals and consistently high decoding accuracy. Exemplary systems may also be applied to non-invasive EEG recordings. Deep brain stimulation is automatically triggered by pain detection via the pain decoder and only lasts for a short period of time to minimize side effects and to enhance treatment efficacy. This system can effectively treat both evoked pain and spontaneously occurring pain that arise at rest. This closed-loop system can also be adapted to other treatment modalities such as ultra-fast drug delivery and electrical or optical or ultrasonic neuromodulation of the brain, spinal cord and/or peripheral nervous system. It will be understood by those of skill in the art that the exemplary embodiments may be adapted to decode pain anticipation by targeting slightly different brain regions.

Closed-loop brain-machine interfaces (BMIs) link neural signals for a sensory or motor event with neuromodulation to restore or enhance brain functions and have the potential to treat neuropsychiatric disorders. BMIs have produced promising results for treating epilepsy and upper or lower motor neuron diseases. However, their application to sensory disorders have remained limited, due to challenges in detecting accurate sensory signals and providing fast and effective behavioral feedback.

Pain represents a unique challenge as well as an opportunity for BMI designs. Chronic pain is one of the most common sensory disorders, and it is defined by discrete episodes of pain that either are evoked by noxious stimuli or occur spontaneously. As discussed above, current treatment options for chronic pain are limited to either scheduled pharmacological interventions or continuous spinal neuromodulation, which do not take into consideration the precise timing of individual pain episodes and result in frequent delays in treatment as well as under- or overtreatment. While, conceptually, a BMI approach is ideally suited to pain management by selectively targeting discrete nociceptive episodes, decoding pain signals remains challenging. Unlike other sensory modalities, there is no single target for pain representations.

Among a distributed network of brain regions that process pain, the primary somatosensory cortex (S1) is known to encode the sensory-discriminative aspect of pain, including the location, timing, and quality of pain, whereas the anterior cingulate cortex (ACC) is known to play a key role in the aversive response to pain in numerous studies across species. As pain is processed in multiple brain regions, an appealing strategy to decode pain is to integrate neural signals from multiple regions, with the S1 and ACC as the most relevant targets. However, as will be understood by those of skill in the art, the exemplary system can also target other brain regions such as, for example, the insular cortex, the thalamus, and the secondary somatosensory cortex (S2) as well.

Current BMI applications primarily rely on neuronal spikes to produce accurate decoded signals. However, individual spikes can be difficult to record faithfully over a prolonged period of time, which is necessary for the clinical management of chronic pain. In contrast, local field potentials (LFPs), which represent the subthreshold synaptic activity from local neuronal populations, are relatively stable in chronic electrophysiological recordings. While their signal stability facilitates clinical applications, LFPs have only recently begun to be used as an alternative to spike activity for population decoding for BMI applications.

In terms of treatment targets, the prefrontal cortex (PFC) is an important center for top-down control of sensory experiences, and multiple lines of evidence from human and animal studies have shown that decreased activity in the PFC contributes to symptoms of chronic pain. Importantly, stimulation of excitatory neurons in the PFC has been demonstrated to produce rapid inhibition of nocifensive withdrawal reflexes and aversive responses to pain without substantial side effects, supporting the PFC as a potential therapeutic arm in BMI design for closed-loop pain control.

According to an exemplary embodiment of the present disclosure, an LFP-based decoding strategy has been developed using recordings from the S1 and ACC, and combined it with optogenetic or electrical stimulation of the PFC to form a multi-region neural interface. As will be discussed in further detail below, studies of the exemplary multi-region LFP based Brain Machine Interface (BMI) of the present disclosure, have shown that the multi-region neural interface of the exemplary embodiments can deliver analgesia for both acute and chronic pain conditions with high sensitivity and specificity over a long period of time.

As shown in FIG. 1, a multi-region LFP-based BMI system 100 according to an exemplary embodiment, has been designed to detect and treat pain in real time, and was tested, as will be described in further described below. The exemplary system 100 is comprised of a plurality of probes 102 for detecting local field potentials (LFPs), a processor 104 including a Pain Decoder Model 106 configured to detect the onset of pain based on the LFPs, and a stimulating device 108 for delivering pain modulation in response to the detected onset of pain. A graphical user interface (GUI), as shown in FIG. 8 and described in further detail below, showing detected LFPs in the brain areas, detection of pain and/or delivery of pain modulation may be displayed to a user via a display. The GUI may also include options for the user to select with respect to LFP detection and pain modulation delivery.

In an exemplary embodiment, probes 102 may be implanted in desired brain regions such as, for example, the ACC and S1 to record LFPs. In an exemplary embodiment, LFPs in the ACC and S1 are detected simultaneously. It will be understood by those of skill in the art, however, that if so desired, users may have the option to select the detection strategy (e.g., based on the ACC, S1, or a combination of both). Although the exemplary embodiments show and describe the probes 102 as being implanted specifically into the ACC and S1 regions, it will be understood by those of skill in the art that probes 102 may be inserted into any desired brain regions of the brain that may produce pain signals. Probes 102 may include, for example, silicone probe arrays but may include any of a variety of probe devices for detecting the LFPs.

LFPs detected by the probes 102 may be processed via the processor 104 and sent to the Pain Decoder Model 106 to detect the onset of pain. In an exemplary embodiment, the Pain Decoder Model may be a Machine Learning model that may be trained based on a state space model, as described below. According to the exemplary embodiment, pain detected by the Pain Decoder Model 106 triggers a neurofeedback activation of the stimulation device 108, which may be placed in a desired brain region to provide stimulation thereof for delivering pain modulation. In an exemplary embodiment, the brain region being treated may include the prelimbic prefrontal cortex (PL-PFC). The stimulation device 108 may include an optical fiber for providing an optogenetic activation of the brain region and/or an electrode configured to provide electrical stimulation thereof. As will be described below, testing of the system 100 confirms that the system 100 is able to provide real-time detection and treatment of pain based on detected LFPs.

The processor 104 may be configured to execute computer-executable instructions for operations from applications that provide functionalities to the system 100. For example, the processor 104 may include instructions for processing the detected LFPs to be sent to the Pain Decoder Model 106. The Pain Decoder Model 106 may include instructions for detecting the onset of pain based on the detected LFPs and, upon detection of the onset of pain, triggers the processor 104 to activate the stimulation device 108. It should be noted, however, that functionalities described with respect to the processor 104 and/or the Pain Decoder Model 106 may also be represented as a separately incorporated component of the system 100, a modular component connected to the processor 104 or as a functionalities achievable via more than one processor 104. For example, the system 100 may be comprised of a network of computing systems, each of which includes one or more of the components and/or which provides one or more of the functionalities described above.

Based on the known role of S1, ACC and PFC in pain processing and regulation, a multi-region, closed-loop neural interface for nociceptive control was developed by using a pain decoder based on concurrent neural signals from the ACC and S1 to trigger therapeutic stimulation of the prelimbic PFC (PL-PFC) in freely behaving rats, as shown in FIGS. 2-3. As shown, silicon probe arrays (Probes 102) were implanted in the rat anterior cingulate cortex (ACC) and primary somatosensory cortex (S1) to record local field potentials (LFPs), which represent the subthreshold local neural activity simultaneously. LFPs are relatively stable in chronic electrophysiological recordings which facilitates clinical application, and they have increasingly been used as an alternative to spike activity for population decoding.

Detected LFP signals were then processed and sent to an automated decoder (Pain Decoder model 106) based on a state space model (SSM) to detect the onset of pain signal. The detected pain onset triggered a neurofeedback via optogenetic or electrical activation of the prelimbic prefrontal cortex (PL-PFC) to deliver pain modulation via, for example, an optical fiber and/or an electrode (stimulation device 108), that was placed in the PL-PFC. It was found that from these signals, as shown in FIG. 4, pain-evoked event-related potentials (ERPs) from the ACC and S1 could be reading identified, indicating that nociceptive signals are contained in these two regions. As described in further detail below, ERP latency was extracted on a trial-by-trial basis, and it was found that the ERP peak latency in the ACC was on average slightly longer than the latency in the S1, suggesting that nociceptive information arrived at the S1 before the ACC, as shown in FIG. 5. These results support the use of LFP signals from S1 and ACC to decode pain.

A model-based unsupervised learning approach was designed to decode pain from multi-region LFP signals. Previous work has shown that spectral features from low gamma (30-50 Hz), high gamma (50-100 Hz), and ultra-high frequency (300-500 Hz) bands are particularly relevant for cortical pain processing. The ultra-high frequency power can be viewed as a proxy for multiunit activity. Thus, frequency-dependent LFP power features were computed and these features were inputted these features to a real-time neural decoder based on a state space model (SSM). As show in FIG. 6, raw LFP signals were processed to compute three band-limited LFP power features for both the ACC channel: {y_{1,k}^{ACC}, y_{2,k}^{ACC}, y_{3,k}^{ACC}} and the S1 channel: {y_{1,k}^{S}1, y_{2,k}^{S}1, y_{3,k}^{S}1}, where the index k denotes the k-th temporal window (bin size 100 ms). MUA: multi-unit activity (300-500 Hz). Two state space models (SSMs) were used to independently infer the latent variables {zₖ^{ACC}} and {zₖ^{S}1} from the LFP features {Yₖ^{ACC}} and {Yₖ^{S}1} of ACC and S1, respectively. The SSM is illustrated by a graphical model with a Markovian structure, in which each node denotes a random variable, and the arrow indicates the statistical dependency between two random variables. A joint detection strategy for the onset of pain signal was utilized. First, the Z-scores were derived from the latent variables {zₖ^{ACC}} and {zₖ^{S}1} (horizontal dashed lines denote the 95% confidence intervals for statistical significance). Next, a moving average cross-correlation function (CCF) was used to compute the correlation between the two Z-score series. The area beyond statistical significance (horizontal dashed lines) was computed to determine the change point. When a pain onset was detected, the decoder automatically triggered optogenetic or DBS stimulation to activate the PL-PFC.

In the presence of a noxious stimulus, the SSM identified a relative change in observed neural activity (Z-scored) in the ACC or S1 from the baseline, and used this change in neural activity as a proxy for the acute pain signal. In order to optimize the specificity of pain detection, a cross-correlation function (CCF) was designed to track temporally coherent changes of pain-encoded LFP features in the S1 and ACC, as the use of concurrent signals from these cortical regions allowed the capture of both sensory and aversive components of pain. This CCF combined the two SSM-inferred Z-scores derived separately from the ACC and S1 LFP features, and optimized the detection performance by adjusting the relative weights of each region's contributions. For online BMI experiments, the CCF-based decoder was used to automatically detect the onset of nociceptive signal to trigger optogenetic or electrical stimulation of the PL-PFC to control pain.

FIG. 7 shows a schematic diagram of a design of the BMI, based on the exemplary system 100, described above. FIG. 8 shows an exemplary GUI of the BMI, which provides options to the user for electing the LFP channels and which allow for visualization of the LFP signals in real-time. As shown in the exemplary GUI, users may have the option to select the detection strategy (based on the ACC, Si, or a combination of both—the CCF method) and change the significance threshold criterion.

The decoding strategy was tested in a set of acute pain assays. First, a noxious (pin prick, PP) or non-noxious (6g von Frey filament, or vF) mechanical stimulus was delivered to the rat's hind paw, while recording LFPs from the contralateral areas of the ACC and S1. As expected, rats showed a higher paw withdrawal rate in response to PP than to vF stimulations. In online experiments, our SSM decoder based on multisite recordings successfully detected the onset of the noxious PP stimulus, as opposed to the non-noxious vF stimulus. In offline experiments, the detection accuracy using LFPs recorded from the ACC, S1, or a combination of both was also compared. In the latter case, the CCF method was used to adjust the weights of the inferred Z-scores from the ACC or S1. It was found that the detection rate for the noxious stimulus (PP) was significantly higher than that of the non-noxious stimulus (6g vF) based on LFPs from either S1, ACC, or a combination of S1 and ACC (the CCF method).

The decoding accuracy based solely on the S1 or on the CCF was higher than the decoding accuracy based solely on the ACC in identifying pain episodes. More importantly, the CCF-based decoding strategy was significantly superior to the strategy based on either the S1 or ACC alone in avoiding false detections (detections in the absence of noxious stimuli). Such high true detection and low false detection rates are critical for the real-life implementation of a BMI system and demonstrates the importance of using multiple regions to optimize sensitivity and specificity in pain decoding.

For online BMI behavior experiments, the decoder was trained using a few calibration trials, and then ran the decoder continuously to automatically detect the onset of pain signals. The false detection rate produced by the CCF-based decoding strategy was compared with the false detection rate produced by single-region decoding strategies. It was found that the multi-region decoding strategy resulted in significant reduction in false detections, compared with detection from either of the two regions alone, further validating the importance of the exemplary multi-region decoding approach.

For therapeutic BMI applications, signal stability is critical. The motivation for using LFP signals is their relative stability over time. To validate this, the reliability of the LFP signals used in the pain decoder of the exemplary embodiment was tested. It was found that the LFP-based strategy of the exemplary embodiment indeed maintained a high level of decoding accuracy over three months, as shown in FIG. 9. FIG. 9 shows a comparison of false detection rates based on LFP decoding strategies using the ACC, S1 and combined (ACC+S1) signals over two sessions, a second session taking place 3 months after the first session, where n=5; P=0.6183 (ACC); p=0.7292 (S1); and p=0.8133 (ACC+S1) using paired t-test. Furthermore, as shown in FIG. 10, when the model parameters derived from the unsupervised learning algorithm trained on day 1 was used, it was found that the same model was able to detect acute pain with high accuracy on day 5, suggesting that the model parameters may not require frequent training or calibration. The first 3 trials were used on Day 1 to train the parameters of the SSM. The same parameters were then used to detect pain on the subsequent 5 days, where n=5; p=0.6888 using one-way ANOVA with repeated measures and post-hoc Tukey's multiple comparison tests. Such signal and model fidelity for pain decoding makes it appealing for real-world applications with chronic neural recordings.

Having established the accuracy, specificity, and reliability of the CCF-based pain decoder of the exemplary embodiment, the exemplary decoder was coupled with optogenetic stimulation of pyramidal neurons of the PL-PFC (using a CaMKII promotor to express channelrhodopsin (ChR2)) to form an analgesic BMI. A conditioned place preference (CPP) assay was used to assess how this BMI could inhibit acute mechanically evoked pain. In the preconditioning phase, animals spent 10 minutes moving freely between two chambers. During conditioning, each chamber was paired with a peripheral (noxious or non-noxious) stimulus in combination with BMI or various control optogenetic neurostimulation protocols. In the testing phase, peripheral stimuli and neurostimulation were removed and the rats were allowed to move freely between the chambers. If the BMI treated pain, the rats should prefer the chamber associated with the BMI. A CPP score was calculated by subtracting the time rats spent in the chamber associated with BMI during the preconditioning phase from the time they spent in the testing phase, as a quantitative measure of the effects of BMI on the reduction of pain-aversive behaviors.

First, noxious PP stimulation coupled with BMI-triggered optogenetic stimulation of the PL-PFC (BMI+PP) was compared with PP coupled with random optogenetic stimulation of the PL-PFC of matching duration and intensity (random neurostimulation+PP). Rats preferred the chamber associated with the BMI, suggesting that it reduced acute mechanical pain. This experiment was then repeated on rats that expressed yellow fluorescent protein (YFP), rather than ChR2, and found that YFP-treated rats did not experience pain relief. A comparison of the CPP scores highlighted the efficacy of the BMI in delivering analgesia. As a positive control, manual activation of the PL-PFC directly following delivery of PP to the paw (manual PL-PFC stimulation+PP) was compared with random PL-PFC stimulation coupled with PP (random neurostimulation+PP). Here, a preference for manual PL-PFC activation in ChR2 rats but not YFP rats was observed.

Results also suggested that the BMI worked as well as precise manual control of the PL-PFC. To confirm this finding, BMI control of the PL-PFC in the presence of PP was compared with manual control of PL-PFC in the presence of PP, and found that rats could not distinguish between these two treatments. Finally, to demonstrate that the effects of the PL-PFC activation delivered by the BMI were specific to pain, the rats' preference for BMI in the presence of a non-noxious vF stimulus (by comparing BMI+6g vF with random PL-PFC activation+6g vF) was examined, and found that the rats did not show a preference for either chamber. These results support the specificity of the BMI in delivering behavioral pain control without substantial side effects.

The exemplary multi-region BMI on acute thermal pain using a Hargreaves test. Infrared (IR) stimulations were delivered at two different intensities—noxious IR 70 and non-noxious IR 10—to the rats' hind paws. It was found that rats withdrew their paws 100% of the time with IR 70 stimulations, compared to <10% of the time with IR 10 stimulations. The multi-region LFP-based pain decoder of the exemplary embodiment successfully detected the onset of acute thermal pain before paw withdrawals with noxious stimulation, in contrast to non-noxious thermal stimulation. Similar to the decoding of mechanical pain, multi-region CCF-based pain decoder showed a significantly lower (<10%) false positive detection rate for non-noxious stimulations than single-region decoding, while maintaining a high (˜80%) detection rate for noxious stimulations. Next, the efficacy of this multi-region neural interface was tested in relieving thermal pain. It was found that the latency to nocifensive paw withdrawal significantly increased in the presence of neurostimulation driven by the exemplary multi-region BMI, and that the exemplary BMI achieved similar effects in reducing nocifensive withdrawals as manually controlled constitutive PL-PFC activation. As expected, control rats that expressed YFP did not demonstrate pain relief, supporting the specificity of BMI control of acute thermal pain.

Next, this closed-loop multi-region neural interface was tested to determine whether the exemplary BMI can also treat chronic pain, using a well-known inflammatory pain model—the Complete Freund's Adjuvant (CFA) model. CFA was injected into the rats' paws contralateral to the implanted recording electrodes. CFA-treated rats demonstrated persistent mechanical allodynia lasting 14 days, as they showed a higher rate of paw withdrawals in response to 6g vF (allodynia-inducing) stimulations than 0.4g vF (non-allodynic) stimulations. The exemplary LFP-based decoding strategy reliably detected the onset of allodynic episodes. Again, the multi-region decoding strategy (using the CCF method) produced a remarkably lower rate of false detections, while maintaining relatively high decoding sensitivity.

Next, the anti-aversive effects of BMI in the CFA model were tested using the CPP assay. The allodynic 6g VF stimulus was paired with the BMI in one chamber and with random PL-PFC activation of matching duration and intensity in the opposite chamber. Rats that expressed ChR2 showed a preference for the chamber associated with BMI; YFP rats, in contrast, did not show any chamber preference. To ensure that the anti-aversive effects of this BMI were specific to pain, the same experiments were repeated using a non-allodynic 0.4 g vF stimulus. In this case, neither ChR2 rats nor YFP rats showed any preference for the BMI treatment.

In addition to hypersensitivity to evoked stimulus, a key pathologic feature of chronic pain is tonic, or spontaneously occurring, pain episodes. Currently, no assays can reliably identify the onset of these episodes, rendering decisions on treatment regimen exceedingly difficult, which results in either delayed treatment, or under- or overtreatment. To test the exemplary strategy, a classic CPP design was used to unmask tonic pain in CFA-treated rats. In this assay, one of the chambers was paired with our multi-region BMI, and the other chamber was paired with random PL-PFC optogenetic stimulation. No peripheral stimuli were given, but the rats were conditioned for a prolonged period of time to unmask tonic pain episodes. The exemplary multi-region decoder was trained using a noxious stimulus (PP). The trained decoder was then allowed to automatically detect tonic pain events in the absence of a peripheral stimulus.

During conditioning, one chamber with the BMI of the exemplary embodiment, which used automated tonic pain detection to trigger optogenetic PL-PFC activation, and the other chamber with random PL-PFC stimulation of matching duration and intensity. It was found that after conditioning, CFA-treated rats preferred the chamber associated with the BMI, indicating that this treatment had a high likelihood of targeting tonic pain episodes, as opposed to random PL-PFC stimulations. YFP-treated control rats did not demonstrate this preference. CPP scores further quantified the efficacy of BMI in reducing the aversive response to chronic tonic pain. These results strongly suggest that the exemplary multi-region neural interface could effectively treat spontaneous pain in a timely fashion. Finally, the anti-allodynic effects of the BMI was tested. It was found that the application of the exemplary BMI significantly reduced mechanical allodynia in CFA-treated rats, further validating the analgesic efficacy of the exemplary neural interface.

While the use of optogenetics provides cell-type specific stimulation, it is not currently available for clinical application. To advance the translational value of the exemplary BMI, optogenetic stimulation of the PL-PFC was replaced with electrical deep brain stimulation (DBS), which has been safely implemented for human use. Electrical stimulation of the PL-PFC was combined with the exemplary multi-region LFP-based decoder to produce a closed-loop BMI-triggered DBS. First, the CPP assay was performed to assess the efficacy of this BMI-triggered DBS in treating acute mechanical pain. It was found that when presented with repeated noxious stimuli (PP), rats preferred the chamber associated with the BMI to the chamber paired with randomly timed DBS, suggesting that the exemplary BMI-triggered DBS produced mechanical pain relief.

Furthermore, this BMI reduced acute thermal pain on the Hargreaves test. Next, the efficacy of this BMI-triggered DBS in treating chronic pain was assessed. It was found that the exemplary system significantly reduced mechanical allodynia in CFA-treated rats. CPP was conducted in the presence of an allodynia-inducing stimulus (6g vF). It was found that when presented with this allodynic stimulus, CFA-treated rats preferred the chamber paired with the BMI to the chamber paired with randomly timed DBS, suggesting that the neural interface reduced pain aversion. Finally, the CPP assay was conducted for spontaneous pain. The exemplary multi-region decoder was trained using the allodynic 6g vF stimulus, and the decoder was subsequently allowed to automatically detect tonic pain episodes and trigger therapeutic DBS during the conditioning phase. It was found that after conditioning, CFA-treated rats preferred the chamber paired with BMI-triggered DBS to randomly delivered DBS. Likewise, when conditioning with BMI-triggered DBS was compared with no DBS, we found that CFA-treated rats preferred the chamber associated with the BMI. These results suggest that BMI-triggered DBS can inhibit tonic pain. To ensure that this BMI-triggered DBS produces no gross side effects, stimulation effects on locomotion were examined and found that it had none.

The study of the multi-region LFP-based neural interface of the system 100, as described above, was engineered as a novel method of analgesic delivery. The exemplary system 100 takes advantage of recordings from multiple brain regions to enhance the specificity of pain decoding; it is stable over time and is compatible with current human electroencephalographic (EEG) or electrocorticographic (ECoG) recording devices. It has been shown that this interface can produce almost instantaneous pain relief. While the use of the exemplary neural interface with optogenetic stimulation of pyramidal PL-PFC neurons supports cell-type specificity and allows it to be used for a variety of mechanistic inquiries, its success with DBS further opens the possibility for clinical application of closed-loop pain control.

There is not one single region in the brain that specifically processes pain information. Instead, different brain regions process different aspects of pain. To meet the challenge of accurate pain detection, in this study a strategy that adapts to this unique multidimensional nature of the pain experience was utilized. In particular, the exemplary neural interface design decodes pain based on neural signals simultaneously recorded from multiple brain regions. Ascending nociceptive signals from the periphery are known to terminate in the ACC and S1. The ACC is well-known to process the affective component of pain across difference species, and neural activity in this region has been previously used to decode the intensity and timing of pain. The S1, meanwhile, provides critical sensory information for pain in a somatotopic manner. Prior studies have further demonstrated that information flow between these two brain regions integrates sensory and affective information to give rise to the overall pain experience. In the testing of the exemplary system 100, the success of the multi-region neural interface in treating acute and chronic pain demonstrates the specificity of decoding based on concurrent signals from the S1 and ACC. At the same time, at the mechanistic level, the results of the above-described testing also validate how these two regions together contribute vitally to the experience of pain.

Another key advancement of the described study is the use of LFPs to decode pain in real time. While spikes provide specific signals at the level of individual neurons, they are less stable over a long period of time in free-moving animals and in humans. In contrast, LFPs provide an alternative solution for neural readout. In the currently described study, LFPs were reliably recorded over a period of three months. This signal stability enables the use of LFPs for BMI applications in chronic recording conditions, which is crucial in the management of chronic pain and similar neuropsychiatric diseases. Remarkably, the exemplary decoding model (Pain Decoder Model 106) remains stable for five days post-training. The robustness of the present model likely results from a combination of signal stability and the use of multi-region decoding, and it shows promise for future clinical translation.

A number of studies have shown that the prelimbic PFC provides pain inhibition through top-down projections as well as projections to other cortical areas. The exemplary embodiment uses this region as the therapeutic arm of the neural interface, as it is one of the few neural structures that can regulate both sensory and affective components of pain, especially in the context of nociceptive inputs. The success of the exemplary BMI in inhibiting both sensory withdrawal and pain aversion validates this choice. There is functional homology between the rodent prelimbic PFC and the dorsolateral PFC in primates, and thus the exemplary neural interface may be adapted to the dorsolateral PFC to provide demand-based treatment in chronic pain patients.

It will be understood by those of skill in the art that while false detections still occur in the above-described study, and they are likely caused by the non-specificity of neuronal firings in the S1 and ACC, and/or by non-stationarity of neural signals in freely behaving rats. However, it has been shown that false detections can be minimized and specificity may be improved by integrating neural activities from two distinct brain regions that have complementary roles in pain processing. This approach to decode pain using signals from multiple brain regions supports the multidimensional nature of the pain experience. Each cortical region such as the S1 or ACC may process a unique aspect of pain, in addition to other behavioral functions. During a pain episode, however, multiple brain regions must activate/inactivate at the same time, and thus a decoder based on activities across multiple nodes of the pain network has a higher likelihood of improving specificity. This decoding approach can be easily extended to incorporate additional brain structures, such as the insular, to further improve decoding specificity. In future studies, neural signals can also be combined with real-time behavioral analyses to achieve even more sensitive and specific pain detection.

The PFC has multiple functions. Thus, nonspecific effects can be expected with neuromodulation treatments deployed by PFC stimulation. Nonspecific side effects are a general issue for neuromodulation, and indeed, they have been observed within existing clinical applications of DBS. There are two strategies to reduce non-specific effects: targeting a highly pain-specific neural structure or group of neurons, or limiting treatment to a defined period of time. Currently, there is not a single known target in the central nervous system that can reliably treat pain without any side effects. In this study, the second strategy was utilized: the exemplary closed-loop, demand-based approach reduces side effects by restricting neuromodulation directly to the duration of the detected pain episodes. As result, gross behavioral deficits were not observed. Future discoveries of neuronal populations with specific pain-regulatory functions may be adapted to the therapeutic interface to further improve treatment specificity and to minimize side effects. At the same time, the exemplary BMI can also be used to facilitate such discoveries.

In conclusion, the multi-region neural interface of the system 100 was designed and tested to show that it produces reliable detection and treatment of pain. The use of stable LFP signals enables chronic use, and also allows our pain decoder to be compatible with ECoG or even EEG recordings. Given the clinical feasibility of EEG or ECOG recordings and DBS, adaptation of our technology could thus open new doors for treatment for patients who suffer from chronic debilitating pain.

Additional improvements to the exemplary closed-loop pain detection and treatment system may include, for example, using neural signals from pain-processing regions such as the ACC and S1 to train the exemplary pain decoder 106. Signals recorded from regions not known to process pain signals may be used as negative controls. The use of such negative controls can further enhance specificity of pain decoding. In a further exemplary embodiment, thresholds for pain decoding in the exemplary algorithm may be adjusted. Thus, distinct models for each individual subject may be produced for high-sensitivity, intermediate-sensitivity or low-sensitivity pain decoding. In these cases, the exemplary decoder 106 and subsequent treatment may allow for different levels of sensitivity and specificity. Such model adjustment can be readily done in preclinical models as well as in pain patients. In the clinical setting, the participating subject can determine which level of sensitivity is best suited for that person's clinical needs.

In addition to the PFC, the exemplary stimulation device 108 of the system 100 may be deployed to target additional brain regions to treat pain. These regions include, but are not limited to, the primary motor cortex, the ACC or periaqueductal gray or thalamus. The exemplary decoding strategy can be applied to other pain-processing brain areas, such as the insular cortex, or the combination of insular cortex and ACC. In another exemplary embodiment, the decoding analysis can be extended from being based on LFP measurements to intracranial EEG signals (potentially accessible to epilepsy patients with depth electrode implant).

As confirmed via the results of the above-described study, a method 200 (as shown in FIG. 11) based on the system 100 may be used to detect and treat pain. In a step 210, neural signals from multiple brain regions of a patient are detected via the probes 102. As described above, these neural signals include LFPs from brain regions which have been shown to indicate pain. In an exemplary embodiment, these brain regions include the ACC and S1. It will be understood by those of skill in the art, however, that neural signals from other brain regions may be additionally, or alternatively, detected and received.

In a step 220, the processor 104 processes the detected LFPs such that the processed LFP data may be sent to the Pain Decoder Model 106, in a step 230. In an exemplary embodiment, the processor may compute frequency dependent power features of the LFPs (step 220), as described above, and inputs these power features to the Pain Decoder Model 106 (step 230). In step 240, the Pain Decoder Model 106 is able to identify relative changes to indicate pain. If pain is detected, a stimulation of a brain region is triggered, in a step 250, to treat the pain. In particular, the processor 104 may trigger the activation of a stimulation device 108 including for example, an optical fiber and/or an electrode to provide optical and/or electrical stimulation of the brain region. As described above, the brain region may include, for example, the prefrontal cortex.

If pain is not detected, the method 200 continues to monitor the brain as described above. In addition, upon the identification of pain and stimulation of the brain region, the method 200 continues to monitor and treat for pain in real time.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

What is claimed is:

1. A computer-implemented method for detecting and treating chronic pain, comprising:
receiving neural signals from multiple brain regions of a patient brain via probes implanted in the multiple brain regions, the neural signals including local field potentials (LFP) of the multiple brain regions;
processing the neural signals and inputting the processed neural signals to a machine learning pain decoder model;
determining, based on the processed neural signals, whether pain is indicated; and
triggering, when pain is indicated via the pain decoder model, a stimulation of a target region of the patient brain based on an indication of the pain.

2. The method of claim 1, wherein processing the neural signals includes computing frequency dependent power features of the local field potentials of the multiple brain regions.

3. The method of claim 2, wherein determining whether pain is indicated includes identifying relative changes in neural activity in the multiple brain regions.

4. The method of claim 1, wherein the multiple brain regions include an anterior cingulate cortex and a primary somatosensory cortex.

5. The method of claim 1, wherein the stimulation of the target region of the patient brain includes an optical stimulation and an electrical stimulation.

6. The method of claim 1, wherein the target region of the patient brain includes a prefrontal cortex.

7. The method of claim 1, wherein the target region of the patient brain includes one of a primary motor cortex, an anterior cingulate cortex, and or periaqueductal gray and thalamus.

8. The method of claim 1, further comprising training the pain decoder model using a state space model based on spectral features from low gamma (30-50 Hz), high gamma (50-100 Hz), and ultra-high frequency (300-500 Hz) bands.

9. A system for treating pain, comprising:
a plurality probes implantable in multiple brain regions of a patient to detect neural signals including local field potentials of the multiple brain regions;
a processing device receiving the neural signals from the multiple brain regions of a patient brain to process the neural signals and input the processed neural signals to a machine learning pain decoder model that is configured to indicate pain; and
a stimulation device implantable in a target region of the patient brain to provide stimulation of the target region based upon an indication of pain.

10. The system of claim 9, wherein the processing device is configured to process the neural signals by computing frequency dependent power features of the local field potentials of the multiple brain regions.

11. The system of claim 10, wherein the pain decoder model is trained to identify relative changes in neural activity in the multiple brain regions.

12. The system of claim 10, wherein the pain decoder model is trained using a state space model based on spectral features from low gamma (30-50 Hz), high gamma (50-100 Hz), and ultra-high frequency (300-500 Hz) bands.

13. The system of claim 9, wherein the processing device is configured to trigger activation of the stimulation device upon an indication of pain.

14. The system of claim 9, wherein the stimulation device is configured to provide one of optical and electrical stimulation of the target region.

15. The system of claim 9, wherein the stimulation device is configured to be implanted in one of a prefrontal cortex, a primary motor cortex, an anterior cingulate cortex, a periaqueductal gray, and thalamus.

16. The system of claim 9, wherein the plurality of probes is configured to be implanted in the multiple brain regions include an anterior cingulate cortex and a primary somatosensory cortex.

17. The system of claim 9, wherein each of the plurality of probes include a silicon probe array.

18. The system of claim 9, further comprising a graphical user interface displaying LFP signals in real-time and providing options to change threshold criterion.

19. A non-transitory computer-readable storage medium including a set of instructions executable by a processor, the set of instructions, when executed by the processor causing the processor to perform operations, comprising:
receiving neural signals from multiple brain regions of a patient, the neural signals including local field potentials (LFP) of the multiple brain regions;
computing frequency dependent power features of the local field potentials of the multiple brain regions;
inputting the power features to a machine learning pain decoder model to identify relative changes in neural activity in the multiple brain regions to indicate pain; and
triggering stimulation of a target region of a brain based on an indication of pain.
